(19) United States
(12) Patent Application Publication
(10) Pub. No.: US 2010/0279903 A1
(43) Pub. Date: Nov. 4, 2010

(54) Metal Salts Of Oxidized Low Molecular Byproduct Polyethylene As Lubricants For PVC

(75) Inventors: Manfred K. Seven, Long Valley, NJ (US); Manfried M. Braun, Stockholm, NJ (US); Vincent M. Clarke, Morris Plains, NJ (US)

Correspondence Address: HONEYWELL/FOX ROTHSCHILD;Patent Services, 101 Columbia Road, Morristown, NJ, 07962, US

(73) Assignee: HONEYWELL INTERNATIONAL INC., Morristown, NJ (US)

(21) Appl. No.: 12/732,853

(22) Filed: Mar. 26, 2010

Related U.S. Application Data
(60) Provisional application No. 61/174,285, filed on Apr. 30, 2009.

(51) Int. Cl. C10M 145/00; C07C 53/00

(52) U.S. Cl. 508/450; 568/959

(57) ABSTRACT

Polymeric salts, lubricant compositions comprising the polymeric salts and processes for producing such polymeric salts. More specifically, low viscosity lubricants for the working surface of an extrusion die during the processing of plastics that require lubricants to render them processable, such as polyvinyl chloride, chlorinated polyvinyl chloride, polyvinylidene chloride and copolymers thereof. A metal base is reacted with acid functional groups formed during oxidation of a wax, thereby forming a polymeric salt, neutralizing the wax and saponifying saponifiable functional groups.

### CROSS-REFERENCE TO RELATED APPLICATIONS

This application is related to and claims the priority benefit of U.S. provisional application No. 61/174,285 filed Apr. 30, 2009, the contents of which are incorporated herein by reference.

### BACKGROUND OF THE INVENTION

1. Field of the Invention

The present invention relates to lubricant compositions and to processes for producing such lubricant compositions. More specifically, the invention pertains to low viscosity lubricant compositions for the working surface of an extrusion die. The lubricants are particularly useful in the processing of plastics that require lubricants to render them proces sable, such as polyvinyl chloride (PVC), chlorinated polyvinyl chloride (CPVC), polyvinylidene chloride (PVDC) and copolymers thereof.

2. Description of the Related Art

For many years, plastics such as PVC, CPVC and PVDC have been widely employed for various uses, including the fabrication of tubing and rigid pressurized piping. Of these materials, PVC is particularly desirable for forming rigid tubing and piping articles because of its low cost and beneficial properties, such as exceptional corrosion resistance to acids, corrosive liquids and gases. PVC tubing also exhibits smooth interior walls for unimpeded flow and reduced sediment buildup. It is non-contaminating, provides fast and reliable solvent-welded connections, offers good pressure-bearing capability, and provides ease of handling and installation. Although PVC is a very versatile polymer exhibiting such desirable properties, PVC base resin is also relatively hard and brittle and is very difficult to process with manufacturing equipment such as extruders, injection molders or other equipment used to process PVC compounds, such as prillers, flakers and pastillators. Accordingly, proper compounding and good lubricant balance are very important in obtaining good machine and end product properties.

It has been known in the art to use lubricant systems as extrusion aids to facilitate the processing of plastics such as PVC, CPVC and PVDC. Lubricants are materials that control the melting point in an extruder/molder to achieve the best processing characteristics and physical properties. Such lubricants may be external, internal or external/internal. External lubricants provide good release from metal surfaces and lubricate between the individual PVC particles and the metal surface. They are normally non-polar molecules, such as alkanes, and are usually paraffin waxes, mineral oils or polyethylene. External lubricants are normally incompatible with PVC, delay fusion and help the PVC slip over the hot melt surfaces of dies, barrels and screws without sticking and contribute to the gloss on the end product surface. They eventually migrate to the melt surface of the PVC, providing lubrication between the polymer and the metal surfaces of the extrusion equipment and are used primarily for processing of rigid PVC in applications where clarity is not a critical factor. External lubricants are prevailingly waxes, with the most conventional being paraffin waxes. Paraffin waxes are separated from crude oil during the production of light lubricating oils. They are clear, odor free and can be refined for food contact.

Internal lubricants provide lubrication at the molecular level between resin particles and reduce the melt viscosity by improving inter-particulate flow when the PVC or other thermoplastic is in molten form. They are normally polar molecules, are usually fatty acids, fatty acid esters or metal esters of fatty acids and are very compatible with PVC. They lower melt viscosity, reduce internal friction and promote fusion. External/internal lubricants provide both external and internal lubrication depending on the combination of chemical groups the lubricants contain. These have chemical groups of both lubricant types and generally have long hydrocarbon chains, along with amide, alcohol, acids and ester groups. Common types of external/internal lubricants used in PVC are fatty acid amides and oxidized polyethylenes. Some of these materials will lubricate as an external lubricant before melting and as internal lubricants after melting. Others will do the reverse. Each of these lubricants should be characterized for its type of lubrication in a given PVC compound.

Previously, fatty acid metal salts were well known for use as lubricants. Such salts are typically formed by the reaction of a fatty acid with a metal oxide, metal carbonate or a metal hydroxide, such as calcium oxide. However, it has also been found that known lubricant compositions blending waxes and such fatty acids or metal salts of fatty acids, undesirably increase the viscosity of the lubricant composition, rendering the lubricant difficult to process and finish on manufacturing equipment. Accordingly, a lower viscosity alternative is desired. The present invention provides a solution to this need in the art.

### SUMMARY OF THE INVENTION

The invention provides a polymeric compound comprising an oxidized wax having metal groups attached thereto, said polymeric compound having an acid number of from about 0.5 mg KOH/gram to about 20 mg KOH/gram and a saponification number of from about 5 mg KOH/gram to about 35 mg KOH/g; and wherein the polymeric compound has a melt viscosity of less than about 450 cps at 140° C.

The invention also provides a process for forming a polymeric compound comprising:

a) combining at least one metal base with a molten non-oxidized wax;

b) at least partially oxidizing the wax, thereby generating acid functional groups; and

c) allowing the metal base to react with the generated acid functional groups, thereby producing a polymeric compound comprising an oxidized wax having metal groups attached thereto, said polymeric compound having an acid number of from about 0.5 mg KOH/gram to about 20 mg KOH/gram and a saponification number of from about 5 mg KOH/gram to about 35 mg KOH/g; and wherein the polymeric compound has a melt viscosity of less than about 450 cps at 140° C.

The invention further provides a process for forming a polymeric compound comprising:

a) providing a wax that is at least partially oxidized, said at least partially oxidized wax having acid functional groups;

b) combining at least one metal base with said at least partially oxidized wax; and

c) allowing the metal base to react with the acid functional groups, thereby producing a polymeric compound comprising an oxidized wax having metal groups attached thereto, said polymeric compound having an acid number of from about 0.5 mg KOH/gram to about 20 mg KOH/gram and a saponification number of from about 5 mg KOH/gram to about 35 mg KOH/g; and wherein the polymeric compound has a melt viscosity of less than about 450 cps at 140° C.

The polymeric salt can be used as an extrusion lubricant, such as a PVC lubricant, by itself or in combination with any other waxes, esters, stabilizers, etc. as may be needed in the art. Such lubricants can be used to provide internal and external lubrication when processing PVC compounds. Internally, it provides good melt blending and low processing temperatures, i.e. reduced shear heat, and externally it prevents the PVC compound from sticking to the metal surfaces of the processing equipment such as extruders, injection molders, prill towers, pastillating equipment, or other equipment used to process PVC compounds. The processes of the invention are exceptionally economical and efficient for producing these products on a large scale.

### DETAILED DESCRIPTION OF THE PREFERRED EMBODIMENTS

It has been unexpectedly found that a polymeric salt compound having a lower viscosity than a wax-metal salt blend may be produced by generating a metal salt in-situ through reaction of a metal base with wax functional groups generated during the oxidation of a wax component. For example, when as low molecular weight polyethylene wax is oxidized, a range of functional groups are generated along the polymer chain including carboxylic acid groups and esters. Upon adding a metal base to this oxidized wax, some or all of the carboxylic acid groups are transformed into the corresponding metal salt, such as, for example, a corresponding calcium salt. Similar results are achieved by dispersing a metal base in a molten, non-oxidized wax, followed by oxidation of the wax. As a result of these steps, the metal base is thereby attached to or directly linked to the carboxylic acid groups and esters generated on the oxidized wax polymer chain, wherein the whole polymer molecule forms the salt. Further, all other saponifiable groups, such as esters, are also converted to salts. This preferably results in full neutralization of the acid groups produced during oxidation as well as full saponification of saponifiable functional groups, thereby producing a compound having low acid and saponification numbers. This resulting polymeric salt is a unique compound that has a lower viscosity compared to simple blends of oxidized waxes and fatty acid metal salts.

It has also been found that the melt viscosity may be further reduced when the wax component which forms the polymeric salts described herein comprises a byproduct wax, which byproduct wax is produced as a byproduct during the polymerization of high density polyethylene. Byproduct waxes formed during the polymerization of high density polyethylene are known, for example, from U.S. Pat. No. 5,728,754, which is incorporated herein by reference. The use of such byproduct waxes in accordance with the invention is exemplified in the Examples below. Such byproduct waxes may alternately be combined with a polymeric salt of the invention to form a multi-component lubricant composition.

The polymeric compound of the invention is a polymeric salt comprising an oxidized wax having metal groups attached thereto. The polymeric salt may be combined with other waxes, such as at least one hydrocarbon wax, esters, or other additives, such as stabilizers, to form a lubricant composition that is useful as an external or internal extrusion lubricant for various polymers, particularly PVC. The phrase “lubricant composition” as used herein is directed to a material comprising one or more polymeric salts as produced herein, which salt is formed by an in-situ reaction of a metal base with carboxylic acid groups generated during the oxidation of the wax component.

The oxidized wax preferably comprises a polyethylene wax, most preferably a low viscosity polyethylene wax. As described herein, a low viscosity polyethylene wax is a substantially linear polymer wax having a melt viscosity of less than or equal to about 100 centipoise at 140° C., a weight average molecular weight of less than about 1500, a number average molecular weight of less than about 800, and a polydispersity index of at least about 3.5. More preferably, the low viscosity polyethylene wax has a viscosity of less than about 75 cps at 140° C., most preferably less than about 50 cps at 140° C. The viscosity values herein are measured using a Brookfield rotational viscometer using techniques that are well known in the art.

In the most preferred embodiments of the invention, a low viscosity polyethylene wax of the invention comprises a byproduct composition recovered during the polymerization of ethylene with a Ziegler-type catalyst, such as a Ziegler-Natta catalyst, via a process conventionally known in the art as the Ziegler slurry polymerization process. In general, the Ziegler slurry polymerization process is used to form high density polyethylene (HDPE) homopolymers or ethylene copolymers, such as ethylene-a-olefin copolymers. A Ziegler-type catalyst is formed by the interaction of the alkyls of Group I-III metals with halides and other derivatives of transition metals in Groups IV-VIII of the Periodic Table of Elements. In a typical catalyst preparation process, the catalyst is prepared from titanium tetrachloride and aluminum trimethyl or a related material. The catalyst may also be impregnated on a support to yield higher efficiency in terms of rate of product per unit of the catalyst. As is well known in the art, suitable catalyst supports are typically inorganic compounds, and is most commonly magnesium chloride. Other suitable supports non-exclusively include those described in, for example, U.S. Pat. Nos. 4,069,169 and 5,409,875, which are incorporated herein by reference. While unsupported Ziegler-Natta catalysts may be used for polymerizations of this type, supported catalysts are generally preferred for polyolefin production because they exhibit much higher activities than non-supported catalysts. In addition to the actual catalyst, a co-catalyst is preferably used. The co-catalyst may generally be any organometallic aluminum-alkyl compound, and preferably comprises triethylaluminum. The function of the co-catalyst is primarily to scavenge impurities in the system, which may terminate polymerization. Typically, the catalyst/co-catalyst pair are TiCl₃ and Al(C₂H₅)₂Cl, or TiCl₄ and Al(C₂H₅)₃. The Ziegler-Natta catalyst, in particular, is synthesized by treating crystalline α-TiCl₃ with [AlCl(C₂H₅)₂]₂, whereby polymerization occurs at special Ti centers located on the exterior of the crystallites.

In a typical Ziegler slurry process, ethylene is fed under low pressure into a reactor that contains liquid hydrocarbon to act as a diluent (i.e. solvent). The diluent is typically a heavy diluent, such as hexane or heptane or a hexane-heptane mixture. The catalyst may be fully prepared and fed into the vessel, continuously or batchwise, or may be prepared in situ by feeding the components directly into the main reactor. The reaction is carried out at some temperatures below 100° C., typically at 70° C., in the absence of oxygen, water, carbon dioxide and the like, all of which reduce the effectiveness of the catalyst. The catalyst remains suspended and the HDPE polymer, as it is formed, becomes precipitated from the solution and a slurry is formed which progressively thickens as the reaction proceeds. Some catalyst remains in the HDPE product. Most of the catalyst residue remains in the byproduct wax polymer/diluent portion.

During polymerization, low molecular weight, wax-like fractions are solubilized in the diluent that is used during polymerization. The diluent acts both as a solvent for the ethylene monomer and as a dispersing agent for the catalyst. The HDPE product of the Ziegler polymerization process is insoluble in the diluent and precipitates, but the byproduct wax produced remains dissolved in the diluent. The recovery of byproduct waxes are generally confined to processes that utilize Ziegler catalysts in heavy diluents, most commonly hexane. Polymerization processes that use light diluents, such as i-butane or propane, do not separate byproduct waxes that can be isolated.

The byproduct wax is a high density polyethylene wax, preferably a polyethylene homopolymer wax that has a density of from about 0.92-0.96 g/cc. The byproduct wax is distinguished from other polyethylene waxes made by direct synthesis from ethylene or made by thermal degradation of high molecular weight polyethylene resins, each of which form polymers of both high and low densities. The byproduct waxes are also generally not recovered from manufacturing processes using light diluents or from other processes such as gas phase polymerization processes or solution polymerization processes.

After completing the Ziegler polymerization of the ethylene, the crude, low viscosity, byproduct wax of the invention is collected. The primary high molecular weight HDPE product is separated by centrifuging from the diluent (e.g. hexane), spent catalyst residue and low molecular weight wax, and the diluent is sent to a diluent recovery unit. Most of the diluent is flashed in the recovery unit for recovery and recycling. The remaining bottom typically contains the byproduct wax, catalyst residue, and possibly residual support compound and small quantities of diluent. Preferably, this mixture is filtered to remove gross contaminants and some, but not all, of the catalyst residue. It is then heated to remove the last traces of remaining diluent (hexane). The crude wax is then filtered using a fine filtration step. This removes 98% of the catalyst residue leaving a wax that still contains oily contaminants and greases, such as oligomers. These interfere with most applications and must be removed in a refining step. They can also be hazardous in that they impart a low flashpoint to the wax.

Refining can be done in a batch type process or in a continuously operating process. There are multiple techniques to further purify the crude byproduct wax, including but not limited to gas stripping, heating or vacuum stripping to remove any residual diluent, holding the melted wax in an unagitated vessel to settle the catalyst and support and decanting off the clarified wax, and filtration of the molten wax through suitable filtration media to remove the catalyst and support. Such techniques are well known in the art. Full refining of the crude wax may be conducted, and may include purification by thin film evaporation. In a preferred refining method, the byproduct wax is heated and subjected to a vacuum at elevated temperatures to strip out the offending oils. The refining equipment used can vary widely from a simple stirred batch tank to a thin film evaporator. Following the refining, an additional filtration step may be conducted if needed. At this point the byproduct wax is free of catalyst residues and offending oils and has undergone considerable property improvements, such as an increased melting point, increased crystallinity due to the removal of oily contaminants, and increased performance consistency as well as increasing the flash point.

The polymeric salts of the invention may be formed following two different processes. In a first process, at least one metal base is combined with a non-oxidized wax, followed by at least partially oxidizing the wax, thereby generating acid functional groups and allowing the metal base to react with the generated acid functional groups, thereby forming a new polymeric compound. In a second process, a wax is at least partially oxidized prior to addition of the metal base, where said oxidation generates acid functional groups, and at least one metal base is thereafter added to the at least partially oxidized wax, allowing the metal base to react with the generated acid functional groups, thereby forming a new polymeric compound.

In accordance with said first process, a non-oxidized wax as described herein is added to a suitable reactor vessel either as a molten or solid wax, preferably being added as solid wax pellets and subsequently melted. A suitable vessel is one that is capable of heating and maintaining the wax components of the lubricant composition at or above its melting temperature. If melting is required, the wax is melted such as by heating said wax component(s) to a temperature of from about 100° C. to about 150° C., more preferably from about 120° C. to about 140° C., preferably with some agitation, to form a wax melt. If melting is not required, the molten wax is preferably maintained as a molten wax by keeping the vessel heated above the melting temperature of said wax.

Thereafter, a metal base is added to the vessel and preferably combined with the molten wax with slight agitation, preferably such that the metal base is substantially homogenously blended with said molten wax prior to oxidation of the wax. In an alternate embodiment, the metal base may be combined with solid wax pellets followed by melting of the wax pellets. The metal base may be added neat to the wax or may be added to the wax either dissolved in or as a slurry in water. Most preferably, the metal base is dissolved or dispersed in water and then metered into the reactor vessel via a metering pump, preferably where the wax has already been melted. The water is preferably flashed off during the base addition, leaving the metal base as a finely divided solid in the molten wax. To ensure complete evaporation of any water, the vessel is continuously maintained at a temperature of 100° C. or greater, thus ensuring that the polymeric compound is completely water-free. The resulting steam may cause foam formation in the melt which is then dissipated. This foaming tendency may be regulated by controlling the addition rate of the base via the metering pump. In other words, the metal base is gradually added to the vessel, allowing the foam to be boiled off in a controlled manner, ensuring that any generated water is evaporated and thereby forming a water-free composition. This procedure will continue until all of the metal base has been reacted and water formation stops. Finally, the molten polymeric salt, or lubricant composition including said molten polymeric salt, may be cooled and held for a finishing step such as prilling or pastillating as is well known in the art. It should be understood that while the methods described herein are preferred, the sequence of steps may be varied as consistent with the invention. For example, it is possible to add all of the metal base at once if using a reactor with a lot of head space to handle potential foam formation.

Suitable metal bases for use herein non-exclusively include metal oxides, metal carbonates and metal hydroxides, which non-exclusively include oxides, carbonates and hydroxides of Group II metals such as calcium, magnesium, barium, zinc, cadmium and lead. Each process of the invention results in said metal groups being attached to the wax polymer chain via reaction with the wax functional groups generated during oxidation. The amount of metal base to be added is calculated on the basis of the final desired acid number and saponification number of the product to be produced. For example, one equivalent of a base per unit acid number could be added in this manner, thereby producing a fully neutralized product following oxidation of the wax. More particularly, the metal base is preferably added to the non-oxidized wax at a concentration of from about 0.5% to about 5.0%, more preferably from about 1.0% to about 3.0% and most preferably from about 1.5% to about 3.0% based on the weight of the non-oxidized wax plus the base. After the metal base and non-oxidized wax are both added to the vessel, the wax is then oxidized by conventional techniques.

In accordance with said second process of the invention, as stated above, a wax is at least partially oxidized prior to addition of the metal base, where said oxidation generates acid functional groups, and at least one metal base is thereafter added to the at least partially oxidized wax, allowing the metal base to react with the generated acid functional groups, thereby forming a new polymeric compound. The metal base is also preferably added to the oxidized wax at a concentration of from about 0.5% to about 5.0%, more preferably from about 1.0% to about 3.0% and most preferably from about 1.5% to about 3.0% based on the weight of the oxidized wax plus the base. Once combined in said vessel, the metal base and oxidized wax react to form the polymeric salt of the invention.

The oxidation process is preferably conducted in a stirred tank reactor. In said process, air or oxygen containing gases are sparged into the reactor, the refined wax is charged to the reactor and then heated to about 130° C. to about 150° C., more preferably from about 142° C. to about 147° C. Air or oxygen containing gases are then admitted to the reactor at an oxygen flow rate of from about 0.5 to about 1.0 standard liters per minute per kg wax (SLM/kg). The pressure in the reactor is preferably controlled at 80-100 psig via a control valve on the exit side. After an initial induction period the oxidation starts with generation of heat. The reactor is preferably cooled by means of an internal cooling coil or an external jacket. During reaction, the reactor is maintained at a temperature higher than the melting point of the wax feedstock, preferably from about 130° C. to about 160° C., more preferably at about 140° C. to about 150° C., and most preferably at about 145° C. Samples may be withdrawn hourly and the acid number is determined. When the desired acid number has been reached, the gas flow is stopped and the reactor is vented to atmospheric pressure. The oxidation is preferably continued until nearly the entire metal base is consumed, or alternately until only a small acid number is determined (e.g., 0.5-1.0 mg KOH/g sample). In the preferred embodiments of the invention, after oxidation, but prior to reaction with the metal base, the wax is oxidized to have an acid number of from about 1 mg KOH/gram to about 40 mg KOH/gram, more preferably from about 5 mg KOH/gram to about 30 mg KOH/gram and most preferably from about 7 mg KOH/gram to about 24 mg KOH/gram. After reaction of a metal base with the carboxylic acid groups produced during oxidation of the wax, the resulting polymeric compound has an acid number of from about 0.5 to about 20 mg KOH/gram, more preferably from about 0.5 to about 10 mg KOH/gram, more preferably from about 0.5 to about 5 mg KOH/gram and most preferably from about 0.5 to about 1.0 mg KOH/gram. The oxidation will also make the wax polar. After oxidation, but prior to reaction with the metal base, the resulting oxidized wax also has a saponification number of from about 8 mg KOH/gram to about 60 mg KOH/gram. After reaction of a metal base with the carboxylic acid groups produced during oxidation of the wax, the resulting polymeric compound has a saponification number of from about 5 mg KOH/gram to about 35 mg KOH/g, more preferably from about 5 mg KOH/gram to about 25 mg KOH/g and most preferably from about 5 mg KOH/gram to about 15 mg KOH/g. Acid and saponification numbers, each having units of mg KOH/gram, are determined by standardized techniques that are well known in the art. Low acid and saponification numbers are preferred and are reflective of complete and efficient neutralization and saponification. Stronger bases than those oxides, carbonates and hydroxides of Group II metals outlined above could be used, such as potassium hydroxide, and would tend to generate lower acid and saponification numbers. However, strong base salts such as those formed from potassium and/or sodium could cause corrosion issues to the fabricator or final user of the polymeric salts and thus may not be desirable.

The oxidized wax or polymeric salt reaction product is then discharged and suitably finished. The polymeric salt prepared in this manner has excellent color, is hard, has a high melting point and has the preferred acid numbers and low viscosity described herein. Alternate conditions may increase the viscosity beyond and above the feedstock viscosity.

Oxidized low viscosity polyethylene waxes of the invention that are produced from a byproduct wax from the Ziegler polymerization process described above are distinguished by the above properties from other waxes which are produced through different methods. Particularly, as illustrated in Table 1 below, not all polyethylene waxes are equivalent. Table 1 provides nuclear magnetic resonance (NMR) data comparing the properties of an oxidized, low viscosity homopolymer byproduct wax, designated in the table as “oxidized CS-600” wax, with an oxidized, low density polyethylene wax designated in Table 1 as “A-C® 629” wax. NMR data is also provided for non-oxidized A-C® 629 wax as well as non-oxidized CS-600 wax. CS-600 is a low viscosity, byproduct wax composition produced by the polymerization of polyethylene with a Ziegler-type catalyst via the Ziegler slurry polymerization process. The CS-600 wax is commercially available from Honeywell International Inc. of Morristown, N.J. As preferred herein, oxidized CS-600 wax is a substantially linear polymer having a melt viscosity of less than or equal to about 100 centipoise at 140° C., an acid number of from about 7 to about 24 mg KOH/gram, a weight average molecular weight of less than about 1500, a number average molecular weight of less than about 800, and a polydispersity index of at least about 3.5.

TABLE 1

| | Non- | | | Non- |
|---|---|---|---|---|
| Branch Distribution | Oxidized | Oxidized | Oxidized | Oxidized |
| (branches/1,000 C) | CS-600 | CS-600 | A-C ® 629 | A-C ® 629 |
| Pendant-CH₃ | 1.5 | 1.4 | 0.8 | 1.1 |
| Ethyl on Quaternary | 0.0 | 0.5 | 5.6 | 5.7 |
| 1,3-Diethyl | 0.4 | 0.7 | 4.9 | 6.2 |
| Regular Ethyl | 1.7 | 1.5 | 2.9 | 3.0 |
| Butyl | 0.4 | 0.3 | 9.5 | 10.5 |
| Pentyl | 0.2 | 0.2 | 2.9 | 3.2 |
| Hexyl and Longer | 44.8 | 41.5 | 8.6 | 9.2 |
| Terminal OH | 0.0 | 0.2 | 2.4 | 2.5 |
| Secondary Alcohol | 0.2 | 1.6 | 3.3 | 0.0 |
| Primary Alcohol | 0.0 | 0.0 | 0.1 | 0.0 |
| Ketone | 0.8 | 7.9 | 5.8 | 0.0 |
| Methyl Ketone or | 0.0 | 2.3 | 2.5 | 0.0 |
| Aldehyde | | | | |
| Chain-End Acid | 0.0 | 3.1 | 3.3 | 0.0 |
| Lactone | 0.0 | 0.9 | 1.0 | 0.0 |
| C═O/OH | 4.00 | 8.00 | 2.13 | 0.00 |
| Aliphatic branches | 48.9 | 46.1 | 35.2 | 38.9 |
| Total branches | 49.9 | 62.0 | 53.7 | 41.4 |

Table 1 above presents a summary of C-13 NMR results for non-oxidized CS-600 feedstock wax, oxidized CS-600 wax, oxidized A-C® 629 wax and non-oxidized A-C® 629 wax (A-C® 6 feedstock wax). The NMR data illustrates that oxidized CS-600 and oxidized A-C® 629 (and their respective non-oxidized feedstocks) are different in their branching distributions and terminal groups. Particularly, the branching in oxidized CS-600 and non-oxidized CS-600 is predominately long chain branching (C₆ or longer). The branching in oxidized A-C® 629 and its non-oxidized feedstock have more short chain branching (C₅ or shorter) than long chain branching. A-C® 629 and its feedstock are terminated with OH groups; oxidized CS-600 and non-oxidized CS-600 are terminated with CH₃ groups. Oxidized CS-600 has a higher C=O/OH ratio than A-C® 629. As shown from the above, the oxidized, low viscosity CS-600 wax is substantially different than a standard oxidized A-C® 629 polyethylene wax, allowing the significant benefits of the invention to be achieved.

As stated above, an oxidized, low viscosity byproduct wax has a viscosity of less than about 100 cps at 140° C., more preferably a viscosity of less than about 75 cps at 140° C., and most preferably less than about 50 cps at 140° C. Accordingly, a polymeric salt of the invention produced with said low viscosity byproduct wax will have a viscosity of less than about 450 cps at 140° C., more preferably less than about 300 cps and most preferably less than about 100 cps at 140° C.

In each of said processes described herein, the metal base and acid functional groups, including carboxylic acid functional groups, generated via oxidation are immediately reacted with each other. Typically, all of the wax is uniformly functionalized, and the reaction results in the attachment or direct linkage of the metal base to the wax polymer chain, resulting in the formation of what is defined herein as a polymeric salt. The polymeric salt reaction product is a new compound and not a blend of a wax and a metal salt. The reaction will also result in continuous saponification of other saponifiable functional groups so that the product is virtually finished as soon as no “free” acid is detected. The metal salt that is formed will depend on the particular selection of metal base. In the preferred embodiment of the invention, the metal base is a metal oxide, metal carbonate or metal hydroxide and comprises a calcium oxide, calcium hydroxide, calcium carbonate, zinc oxide or zinc carbonate. Most preferably the metal base comprises calcium oxide or calcium hydroxide. The metal base may also react with any other functional groups in the oxidized wax, such as primarily esters and acid anhydrides, and to a smaller degree, peroxides, ketones and aldehydes.

As stated above, the polymeric salt of the invention can be used as an extrusion lubricant, such as a PVC lubricant, by itself or in combination with any other waxes, esters, stabilizers, etc. as may be needed in the art. In a preferred embodiment, at least one polymeric salt of the invention is combined with at least one hydrocarbon wax to form a lubricant composition. For the purposes of the invention, hydrocarbon waxes include (non-oxidized) polyethylene waxes, microcrystalline waxes, paraffin waxes, alpha-olefin waxes and Fischer-Tropsch waxes. Including said optional hydrocarbon wax or waxes, the polymeric salt component preferably comprises from about 5% to about 95% by weight of said lubricant composition, more preferably from about 25% to about 75% by weight and most preferably comprises about 50% by weight of said lubricant composition. Accordingly, the one or more hydrocarbon waxes preferably comprises from about 5% to about 95% by weight of said lubricant composition, more preferably from about 25% to about 75% by weight and most preferably comprises about 50% by weight of said lubricant composition.

Lubricant compositions formed herein may also include additives, such as pigments or stabilizers, as is well known in the art, as described in U.S. Pat. No. 4,544,694, the full disclosure of which is incorporated herein by reference. The polymeric salts may also be combined or blended with other lubricating ingredients, such as fatty alcohol or fatty acid ester, after finishing the metal salts in the same vessel without requiring any intermediate finishing steps. Suitable fatty alcohols non-exclusively include, for example, stearyl alcohol, lauryl alcohol or combinations thereof. Suitable fatty acid esters non-exclusively include glycerol fatty acid esters, such as glycerol monostearate. Such fatty alcohols/fatty acid esters have an internal lubricating effect that may enhance the lubrication properties of the polymeric salt. If added, such additional lubricating ingredients are preferably added to comprise a quantity of about 5% to about 15% by weight of said polymeric salt, more preferably from about 7.5% to about 12.5% by weight and most preferably from about 9% to about 10% by weight of said polymeric salt.

The resulting compounds and compositions of matter including said compounds have been found to be excellent internal or external extrusion lubricants for the extrusion of thermoplastic polymer materials, particularly vinyl polymers such as PVC, CPVC, PVDC and copolymers thereof. See, for example, U.S. Pat. No. 5,426,144, the disclosure of which is incorporated herein by reference, which teaches alternate extrusion lubricant compositions. See also, for example, U.S. Pat. No. 4,030,328 which teaches a process for the continuous lubrication of the working surfaces of an extrusion die. In said embodiment, the polymeric salts of the invention may be blended with a thermoplastic polymeric material to form a mixture, and subsequently extruding said mixture. In addition to their use as extrusion aids, the polymeric salts and lubricant compositions of this invention may also be used as nucleating agents, metal powder lubricants, mold release agents, heat stabilizers and like applications where materials such as relatively pure calcium stearate are currently being used. As a lubricant, an effective amount of polymeric salt or lubricant composition for lubricating the vinyl polymer is used. Typically, the salt or lubricant composition is present in an amount of about 0.01 to about 10 parts by weight per 100 parts of vinyl polymer. Preferably, the salt or lubricant composition is present in an amount of about 0.05 to about 5 parts by weight per 100 parts of vinyl polymer, more preferably in an amount of about 0.05 to about 1 part by weight per 100 parts of vinyl polymer, and most preferably in an amount of about 0.1 to about 1 part by weight per 100 parts of vinyl polymer.

The invention is more specifically described with reference to the examples below wherein all parts and percentages are by weight unless otherwise specified. The examples serve to illustrate the invention and the present invention should not be construed to be limited thereto.

### EXAMPLES

### Example 1

850 g of a byproduct wax having the following properties: 50 cps viscosity at 140° C., 116.3° C. drop point, 3.2 dmm hardness penetration at 25° C. and 0.935 g/cc density at 23° C. were charged to a 2 liter resin flask equipped with a speed controlled overhead stirrer using a U shaped agitator, a PID controlled heating mantle, an internal thermocouple and a nitrogen sweep. The melted wax was brought to 150° C. at which point calcium oxide addition was begun in the form of a slurry consisting of 13.6 grams of calcium oxide in 130 g water. The slurry was added in small increments from a stirred tank over a 1 hour period during which time the temperature of the mixture was kept between 140° C.-150° C. The product was then dehydrated for a period of 20 minutes at 150° C. at which point the oxidation was started.

The oxygen was delivered to a point below the agitator via a dip tube terminating in a stainless steel frit. The oxidation was carried out at 150° C., 600 rpm mixing speed and 0.8 SLM/kg flow of oxygen. The reaction was sampled hourly and followed by titration to determine the acid number. After 3 hours, a slight amount of acid was detected indicating that the base had effectively been fully neutralized. The reaction was halted at this point and the product discharged and cooled. Analysis of the finished product showed a final acid number of 1.7 mg KOH per gram, viscosity equal to 34 cps @ 140° C., a drop point of 103.8° C., and a hardness of 3.5 dmm @ 25° C.

### Example 2

Two polymeric salt samples produced in accordance with the process described herein were compared with a lubricant blend as produced in accordance with the teachings of pending application Ser. No. 11/589,486, the disclosure of which is incorporated herein by reference in its entirety. All three of the samples were produced utilizing a byproduct polyethylene wax produced by the Ziegler polymerization process. In Inventive Sample A, the byproduct wax was first oxidized and then calcium oxide was added. In Inventive Sample B, the byproduct wax was oxidized after addition of calcium oxide. In the Comparative Sample according to Ser. No. 11/589,486, the byproduct wax was oxidized prior to addition of calcium oxide and prior to reacting calcium oxide with stearic acid. In addition to the oxidized byproduct wax, the Comparative Sample composition also included an ester, glycerol monostearate, free stearic acid and calcium stearate. An analysis of the samples identified the properties presented in Table 2 below. Specifically, Inventive Samples A and B show much lower saponification values as compared to the Comparative Sample. This is also reflected and confirmed by the large difference in acid numbers between the inventive and comparative compositions. These substantial differences illustrate that it is unlikely that much of any saponification/neutralization with calcium in the Ser. No. 11/589,486 blend occurred with respect to the oxidized byproduct wax.

TABLE 2
Calcium Salts of Oxidized Byproduct PE Wax

| | | | Pending |
|---|---|---|---|
| | | | Application |
| | | Reverse | 11/589,486 |
| | | Process* | Sample |
| | Inventive | Inventive | Comparative |
| | Sample A | Sample B | Sample |
| Acid # of Feed | 16 | NA | — |
| Material | | | |
| Approx. | Full | Full | — |
| Neutralization | | | |

Product Properties

| | | | | |
|---|---|---|---|---|
| Residual Acid# | mg KOH/g | 0.6 | 1.7 | 20.6 |
| Viscosity | cps @ 140° C. | 51 | 34 | 121 |
| Drop Point | ° C. | 104.6 | 103.8 | NA |
| Hardness | dmm @ RT | 3.4 | 3.5 | NA |
| SAP Number | mg KOH/g | 9.4-12.2 | 9.6 | 37.2 |

*Calcium oxide dispersed in molten wax followed by oxidation.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above, and all equivalents thereto.

What is claimed is:

1. A polymeric compound comprising an oxidized wax having metal groups attached thereto, said polymeric compound having an acid number of from about 0.5 mg KOH/gram to about 20 mg KOH/gram and a saponification number of from about 5 mg KOH/gram to about 35 mg KOH/g; and wherein the polymeric compound has a melt viscosity of less than about 450 cps at 140° C.

2. The polymeric compound of claim 1 wherein said oxidized wax comprises a substantially linear polymer and having a weight average molecular weight of about 1500 or less, a number average molecular weight of about 800 or less, and a polydispersity index of at least about 3.5.

3. The polymeric compound of claim 1 wherein said wax comprises a byproduct wax produced as a byproduct of the polymerization of ethylene with a Ziegler-type catalyst.

4. The polymeric compound of claim 3 wherein said byproduct wax has a melt viscosity of about 100 centipoise or less at 140° C.

5. The polymeric compound of claim 1 wherein said compound has a viscosity of about 100 cps or less at 140° C.

6. The polymeric compound of claim 1 wherein said wax has an acid number of from about 0.5 mg KOH/gram to about 15 mg KOH/gram and a saponification number of from about 7 mg KOH/gram to about 25 mg KOH/g.

7. The polymeric compound of claim 1 wherein said wax has a melting point of from about 40° C. to about 150° C. as determined by ASTM D-3954, a hardness of from about 0.1 dmm to about 10 dmm at 25° C. as determined by ASTM D-5, and a density of from about 0.92 g/cc to about 0.96 g/cc at 23° C. as determined by ASTM D-1505.

8. The polymeric compound of claim 1 wherein said polymeric compound comprises a fully neutralized wax.

9. A composition comprising the polymeric compound of claim 1 and further comprising at least one polyolefin wax, oxidized polyolefin wax, Fischer-Tropsch wax, beeswax, Chinese wax, shellac wax, spermaceti wax, wool wax, bayberry wax, candelilla wax, carnauba wax, castor wax, esparto wax, Japan wax, jojoba oil wax, ouricury wax, rice bran wax, soy wax, ceresin wax, montan wax, ozocerite, peat wax, paraffin wax, a microcrystalline wax, a polyethylene wax, polypropylene wax, wax grade polytetrafluoroethylene, polytetrafluoroethylene-modified waxes, alpha-olefin wax, amide wax, stearamide wax, or combination of two or more thereof.

10. A process for forming a polymeric compound comprising:
a) combining at least one metal base with a molten non-oxidized wax;
b) at least partially oxidizing the wax, thereby generating acid functional groups; and
c) allowing the metal base to react with the generated acid functional groups, thereby producing a polymeric compound comprising an oxidized wax having metal groups attached thereto, said polymeric compound having an acid number of from about 0.5 mg KOH/gram to about 20 mg KOH/gram and a saponification number of from about 5 mg KOH/gram to about 35 mg KOH/g; and wherein the polymeric compound has a melt viscosity of less than about 450 cps at 140° C.

11. The process of claim 10 wherein said oxidized wax comprises a substantially linear polymer, said wax having a weight average molecular weight about 1500 or less, a number average molecular weight of about 800 or less, and a polydispersity index of at least about 3.5.

12. The process of claim 10 wherein said wax comprises a byproduct wax produced as a byproduct of the polymerization of ethylene with a Ziegler-type catalyst.

13. The process of claim 10 wherein said metal base is added to said wax as an aqueous slurry comprising water, which water is evaporated to provide a water-free polymeric compound.

14. The process of claim 13 wherein said water is evaporated prior to oxidizing said wax.

15. The process of claim 10 wherein the metal base is substantially homogenously blended with said molten wax prior to oxidation of the wax.

16. The process of claim 10 further comprising:
a) blending said polymeric compound with a thermoplastic polymeric material to form a mixture, and subsequently extruding said mixture; or
b) utilizing said polymeric compound as an extrusion lubricant.

17. A process for forming a polymeric compound comprising:
a) providing a wax that is at least partially oxidized, said at least partially oxidized wax having acid functional groups;
b) combining at least one metal base with said at least partially oxidized wax; and
c) allowing the metal base to react with the acid functional groups, thereby producing a polymeric compound comprising an oxidized wax having metal groups attached thereto, said polymeric compound having an acid number of from about 0.5 mg KOH/gram to about 20 mg KOH/gram and a saponification number of from about 5 mg KOH/gram to about 35 mg KOH/g; and wherein the polymeric compound has a melt viscosity of less than about 450 cps at 140° C.

18. The process of claim 17 wherein said oxidized wax comprises a substantially linear polymer, said wax having a weight average molecular weight about 1500 or less, a number average molecular weight of about 800 or less, and a polydispersity index of at least about 3.5.

19. The process of claim 17 wherein said wax comprises a byproduct wax produced as a byproduct of the polymerization of ethylene with a Ziegler-type catalyst.

20. The process of claim 17 wherein said metal base is added to said oxidized wax as an aqueous slurry comprising water, which water is evaporated to provide a water-free polymeric compound.
